(19) United States
(12) Patent Application Publication
(10) Pub. No.: US 2021/0330263 A1
(43) Pub. Date: Oct. 28, 2021

(54) SYSTEM FOR DETECTING BIOSIGNALS

(71) Applicant: MENTALAB GMBH, Berlin (DE)

(72) Inventor: Sebastian HERBERGER, Munich (DE)

(21) Appl. No.: 17/273,706

(22) PCT Filed: Sep. 6, 2019
(86) PCT No.: PCT/EP2019/073813

§ 371 Date: Mar. 4, 2021

(30) Foreign Application Priority Data
Sep. 7, 2018 (DE) 10 2018 007 124.4

(51) Int. Cl. A61B 5/00; G06K 9/03; A61B 5/113; A61B 5/08; A61B 5/346

(52) U.S. Cl. CPC A61B 5/6833; G06K 9/033; A61B 5/1135; A61B 5/0809; A61B 2562/06; A61B 5/0006; A61B 2562/225; A61B 2562/0219; A61B 2562/0261; A61B 5/346

(57) ABSTRACT

The invention relates to a system for detecting biosignals, comprising a sensor unit and a patch that can be attached to the body and has electrodes and conductor tracks. The sensor unit and the patch can be connected to one another via a connector arranged on the patch in such a way that an electrical connection is produced and the sensor unit is held on the body via the patch. The sensor unit has a plurality of contact elements for electrically contacting the connector and/or the patch. According to the invention, at least two contact elements of the sensor element are conductively connected to one another via a conductor track of the patch by connecting the patch to the sensor unit.

The present invention relates to a system for detecting biosignals having a sensor unit and a patch that can be attached to the body and has electrodes and conductor tracks, wherein the sensor unit and the patch are connectable to one another via a connector arranged at the patch such that an electrical connection is established and the sensor unit is held at the body via the patch.

Such systems permit a substantially more comfortable detection of biosignals such as ECGs since, on the one hand, the position of the electrodes with respect to one another is defined by the patch and, on the other hand, the sensor unit can be worn on the body, which in particular substantially improves long-term measurements. The sensor unit is here preferably held solely by the adhesive force of the patch on the patient.

Such systems are known, for example, from US 2015/0164324 A1. The connector and the patch are there designed in an embodiment as a disposable to which the sensor unit is connected to measure biosignals. The electrical connection of the sensor unit takes place by electrical contacts of the connector that in turn contact contact regions of the conductor tracks of the patch.

A further such a system is known from EP 1 979 040 B1. The mechanical connection takes place by a latching of the sensor unit into arms of the connector that laterally grip around the housing of the sensor unit. The elastic connection of the sensor unit takes place by electrical contacts of the connector that in turn contact contact regions of the conductor tracks of the patch. The electrical contacts of the connector can here contact conductor tracks arranged at a rear side of the patch or can take place by conductor tracks arranged at a tab folded beneath the connector.

Documents WO 2015/002935 A2 and WO 2016/067101 A2 show further systems for detecting biosignals having a sensor unit and a patch that can be attached to the body and has electrodes and conductor tracks, wherein the sensor unit and the patch are connectable to one another via a connector arranged at the patch such that an electrical connection is established and the sensor unit is held at the body via the patch.

It is the object of the present invention to provide an improved system for detecting biosignals.

This object is achieved in a plurality of independent aspects by the systems described in more detail in the following.

In accordance with a first independent aspect, the present invention comprises a system for detecting biosignals having a sensor unit and a patch that can be attached to the body and has electrodes and conductor tracks, wherein the sensor unit and the patch are connectable to one another via a connector arranged at the patch such that an electrical connection is established and the sensor unit is held at the body via the patch, wherein the sensor unit has a plurality of contact elements for an electrical contact of the connector and/or of the patch. The first aspect is characterized in that at least two contact elements of the sensor element are conductively connected to one another via a conductor track of the patch by the connection of the patch to the sensor unit. The patch therefore in particular closes a circuit when it is connected to the sensor unit.

The connection of two contact elements of the sensor element via a conductor track of the patch can be used for different tasks.

For example, the sensor unit in accordance with the second aspect described in the following can recognize by it that a patch is connected to the sensor unit and can, for example, automatically switch itself on and/or start the recording.

Alternatively or additionally, the connection of two contact elements of the sensor element via a conductor track of the patch can be used to recognize the patch type that is connected to the sensor unit. For example, by a corresponding design of their conductor tracks, different patch types can establish different connections between the contact elements of the sensor unit and can hereby be recognizable.

The sensor unit can be designed such that it stores and/or transmits the patch type together with the sensor signals, when said patch type is recognized by it.

Alternatively or additionally, the sensor unit can be designed such that it automatically switches into an operating mode matching the respective patch type on the basis of the recognized patch type.

In accordance with a second independent aspect, the present invention relates to a system for detecting biosignals having a sensor unit and a patch that can be attached to the body and has electrodes and conductor tracks, wherein the sensor unit and the patch are connectable to one another via a connector arranged at the patch such that an electrical connection is established and the sensor unit is held at the body via the patch. The second aspect is characterized in that the sensor unit recognizes that it has been connected to a patch and automatically switches itself on as a response. The user therefore no longer has to switch the sensor unit on separately. The prevents the user from accidentally not switching the sensor on and therefore increases operating safety particularly on operation by the patient himself.

In a possible embodiment of the present invention, the patch and the sensor unit are set up so that a circuit of the sensor unit is closed by the connecting of the patch to the sensor unit. The sensor unit preferably recognizes from this that it has been connected to a patch.

In a possible embodiment of the present invention, the sensor unit has a plurality of contact elements for an electrical contact of the connector and/or of the patch. The circuit is preferably closed in that at least two contact elements of the sensor element are conductively connected to one another by the connection of the patch to the sensor unit, in particular by a conductor track of the patch. The patch therefore preferably short circuits two contact elements of the sensor element. The sensor unit preferably recognizes from this that it has been connected to a patch.

In a possible embodiment of the present invention, the recording of signals starts automatically when the sensor unit recognizes that it has been connected to a patch. The sensor unit therefore not only automatically switches itself on, but also automatically starts the recording. Operating safety is hereby again increased.

In a possible embodiment of the present invention, the recording of signals ends as soon as the sensor unit recognizes that it is no longer connected to a patch.

The sensor unit preferably recognizes from this that a circuit that is closed with a connected patch is opened so that it is no longer connected to a patch.

In accordance with a third independent aspect, the present invention comprises a system for detecting biosignals having a sensor unit and a patch that can be attached to the body and has electrodes and conductor tracks, wherein the sensor unit and the patch are connectable to one another via a connector arranged at the patch such that an electrical connection is established and the sensor unit is held at the body via the patch, wherein the sensor unit has at least one first sensor that is connected to the electrodes of the patch by the connection of the patch to the sensor unit and detects biosignals by means of these electrodes. The third aspect is characterized in that the sensor unit furthermore has at least one second sensor for detecting position data and/or rotation data and/or acceleration data.

The data of a position sensor and/or rotation sensor and/or acceleration sensor can be used in a variety of ways to control the sensor unit and/or for an improved evaluation of the data.

In a possible embodiment of the present invention, the sensor unit has at least one operating mode in which the data of the first and second sensors are used together to monitor a patient.

In a possible embodiment of the present invention, the data of the second sensor can be used to recognize artifacts in the data of the first sensor. For example, fast movements of the patient often generate interference signals by the associated mechanical strains on the patch, said interference signals being able to be recognized as such by the evaluation of the data of the second sensor.

In a possible embodiment of the present invention, the data of the second sensor are used to recognize user inputs. The data can in particular be used to recognize a single or multiple knocking on the sensor unit. Such a knocking can be used by the patient, for example, to flag time episodes for later analysis in which he determines specific symptoms such as palpitations.

The data of the first and second sensors can be stored and/or transmitted together by the sensor unit for the above-described possible embodiments of the present invention to then be available for a corresponding evaluation that does not, however, necessarily have to take place by the sensor unit itself. The data of the first and second sensors are preferably stored and/or transmitted by the sensor unit such that a temporal correlation between the data can be established.

In a possible embodiment of the present invention, the system comprises an evaluation unit to which signals of the first and/or second sensors detected by the sensor unit and/or stored by the sensor unit are transmitted for evaluation. The above-described evaluations preferably take place on this evaluation unit.

Alternatively or additionally, the sensor unit can partially or completely evaluate the data of the first and/or second sensors itself. The sensor unit preferably partially or completely evaluates at least the data of the second sensor itself and/or transmits the results of this evaluation together with the first data.

In a possible embodiment, the data of the second sensor can also be used to control the sensor unit, for example to switch the sensor unit on and/or off and/or to change the recording mode. The user can, for example, hereby initiate a more exact detection of the signal of the first sensor as part of an ongoing monitoring in that he knocks once or multiple times on the sensor unit.

In accordance with a fourth independent aspect, the present invention comprises a system for detecting biosignals having a sensor unit and a patch that can be attached to the body and has electrodes and conductor tracks, wherein the sensor unit and the patch are connectable to one another via a connector arranged at the patch such that an electrical connection is established and the sensor unit is held at the body via the patch, wherein the sensor unit has at least one first sensor that is connected to the electrodes of the patch by the connection of the patch to the sensor unit and detects biosignals by means of these electrodes. The fourth aspect is characterized in that the system, and in particular the sensor unit, has a function for detecting breathing activity, in particular the breathing frequency. Further clinically relevant data are hereby generated.

In a possible embodiment of the present invention, the detection of breathing activity takes place by detection of an impedance change of ECG leads by means of the patch. The impedance is preferably measured by applying a voltage between two electrodes of the patch and measuring the resulting current. The sensor unit therefore, on the one hand, has a function for applying the voltage and, on the other hand, a function for measuring the current.

In a possible embodiment of the present invention, the detection of breathing activity takes place by detection of a periodic changes of the ECG vector by means of the patch.

In a possible embodiment of the present invention, the detection of breathing activity takes place by detection of the data of at least one strain gauge integrated in the patch.

In a possible embodiment of the present invention, the detection of breathing activity takes place by detection of a movement of the thorax by a second sensor arranged in the sensor unit for detecting position data and/or rotation data and/or acceleration data.

A plurality or all of these possibilities can be combined with one another.

The detection of the breathing frequency from the signals measured by the sensor unit can take place by an evaluation unit of the system separate from the sensor unit and/or by the sensor unit itself.

In this process, the data of the first sensor can be detected, stored, and/or transmitted by the sensor unit together with further sensor data from which breathing activity, in particular the breathing frequency, is detected to then be available for a corresponding evaluation. The data of the first and second sensor are preferably stored and/or transmitted by the sensor units such that a temporal correlation between the data can be established.

In accordance with a fifth independent aspect, the present invention comprises a system for detecting biosignals having a sensor unit and a patch that can be attached to the body and has electrodes and conductor tracks, wherein the sensor unit and the patch are connectable to one another via a connector arranged at the patch such that an electrical connection is established and the sensor unit is held at the body via the patch. The fifth aspect is characterized in that the sensor unit has at least two different operating modes that work with different patches connectable to the sensor unit.

In a possible embodiment of the present invention, the sensor unit automatically recognizes the desired operating mode with reference to the patch connected to it.

In a possible embodiment of the present invention, the sensor unit has a plurality of contact elements for electrical contact with the patch, with the sensor unit recognizing the desired operating mode by evaluation of signals at the contact elements.

The patch and/or the operating mode can, for example, be recognized by the evaluation of non-used contact elements. The patch can either leave such non-used contact elements free or can short circuit them to one another or to a further contact. Both can be detected by the sensor unit.

Alternatively or additionally, the sensor unit can recognize the operating mode with reference to the type of the applied biosignals and can thus, for example, distinguish between the recording of an EEG and an ECG.

In a possible embodiment of the present invention, the operating modes and/or patches differ at least with respect to the number and/or the geometrical arrangement of the electrodes. Alternatively or additionally, the operating modes can differ by the type of detected biosignals.

In a possible embodiment of the present invention, the operating modes differ by the duration and/or frequency and/or accuracy of the detection of the biosignals and/or the type of the evaluation and/or storage and/or transmission of the detected data.

In accordance with a sixth independent aspect, the present invention relates to a system for detecting biosignals having a sensor unit and a patch that can be attached to the body and has electrodes and conductor tracks, wherein the sensor unit and the patch are connectable to one another via a connector arranged at the patch such that an electrical connection is established and the sensor unit is held at the body via the patch. The sixth aspect is characterized in that the sensor unit can work with at least two different patch types and automatically recognizes the respectively connected patch type.

In a possible embodiment of the present invention, the sensor unit has a plurality of contact elements for electrical contact with the patch, with the sensor unit recognizing the patch type by evaluation of signals at the contact elements.

For example, different patch types can establish different connections between the contact elements of the sensor unit and can hereby be recognizable by a corresponding design of their conductor tracks.

The sensor unit can be designed such that it stores and/or transmits the patch type together with the sensor signals, when said patch type is recognized by it.

Alternatively or additionally, the sensor unit can be designed such that it automatically switches into an operating mode matching the respective patch type on the basis of the recognized patch type.

The above-described aspects of the present invention are initially independent of one another. However, two or more of these aspects are preferably implemented in combination. The present invention here comprises all the conceivable combinations of the above-described six independent aspects.

Possible embodiments of the independent aspects and any desired combinations of these aspects will be described in more detail in the following.

In a possible embodiment of the present invention, the sensor unit has at least one operating mode in which an ECG is recorded, with the sensor unit preferably having at least two operating modes in which different types of ECGs are recorded, with the different types of ECGs preferably differing by the number of the electrodes and/or by the duration and/or frequency and/or accuracy of the detection of the biosignals and/or the type of evaluation and/or storage and/or transmission of the detected data.

In a possible embodiment of the present invention, the sensor unit has at least one operating mode in which an EEG is recorded, with the sensor unit preferably having at least two operating modes in which different types of EEGs are recorded, with the different types of EEGs preferably differing by the number of the electrodes and/or by the duration and/or frequency and/or accuracy of the detection of the biosignals and/or the type of evaluation and/or storage and/or transmission of the detected data.

In a possible embodiment of the present invention, the sensor unit detects at least one of the following biosignals in at least one operating mode and preferably in a plurality of operating modes: ECG, EEG, EOG and/or EMG.

In a possible embodiment of the present invention, the sensor unit has one or more wireless interfaces for data transmission.

In a possible embodiment of the present invention, the sensor unit has a memory for storing detected data.

In a possible embodiment of the present invention, the sensor unit has a function for compressing and/or evaluating detected raw data.

In a possible embodiment of the present invention, the sensor unit is releasably connectable to the connector by means of a rotation of the housing relative to the connector. This permits a 1-point fixing of the sensor unit to the connector that can preferably be established using one hand and is further preferably also releasable with one hand again. The connection via a rotational movement furthermore has the advantage that a relatively small base surface of the connector is sufficient for the mechanical connection to the housing.

Provision is made in a possible embodiment that the connector has a mechanical connection region that is releasably connectable to a mechanical connection region of the sensor unit by a rotational movement. The mechanical connection regions preferably only lock at one another in a single defined rotational position. This ensures a safe electrical contact since the corresponding electrical contact elements are hereby uniquely spatially associated with one another in the locked position.

Provision is made in a possible embodiment that the mechanical connection regions respectively surround an electrical connection region, with the mechanical connection regions preferably substantially circularly surrounding the electrical connection region. A safe electrical connection is hereby ensured by the establishing of the mechanical connection.

Provision is made in a possible embodiment that the connector only establishes the mechanical connection to the sensor unit and the electrical contact takes place directly between the sensor unit and the patch. This has the advantage that the connector does not require any electrical contact. It can therefore, for example, be produced completely from plastic, for example as an injection molded part, in particular as an injection molded part produced in one piece. The connector can hereby be produced substantially cheaper.

In a possible embodiment, the connector is shaped such that at least one contact surface of a conductor track of the patch is accessible from the sensor unit and can in particular be contacted by a contact pin of the sensor unit.

In a possible embodiment, the connector is arranged on a region of the patch designed as a folded-over tab. The mechanical connection between the connector and the patch preferably takes place via the tab here. The arrangement of the connector at the tab has the advantage that the size and shape of the part of the plastic not folded over can be selected independently of the size and shape of the connector.

In a possible embodiment, a counter-element arranged on a side of the tab remote from the connector is connected to the part of the patch not folded over.

The patch having the connector arranged on the patch preferably forms a disposable.

The patch preferably has at least two electrodes. In a possible embodiment, the patch comprises exactly two electrodes.

The patch preferably has an adhesive layer on its side facing the body, said adhesive layer preferably being covered via a removable protective liner.

The present invention furthermore comprises the respective sensor units for the above-described systems.

The present invention furthermore comprises a patch for detecting biosignals, in particular for one of the systems such as were described above, having electrodes and conductor tracks, wherein the patch has a connector via which it is connectable to a sensor unit such that an electrical connection is established and the sensor unit is held at the body via the patch, wherein the patch and/or the connector has/have a plurality of contact surfaces for the electrical contact with contact elements of the sensor unit, with at least two contact surfaces being conductively connected to one another. The advantages that were already described in more detail above with respect to the second aspect can hereby be achieved.

In a possible embodiment of the present invention, the contacts surfaces conductively connected to one another are not connected to one of the electrodes. Alternatively, the contact surfaces conductively connected to one another are only connected to one of the electrodes.

The present invention furthermore relates to a set of at least two different patches that are connectable to the same sensor unit for one of the systems such as has been described above.

The present invention will now be described in more detail with reference to embodiments and drawings.

There are shown:

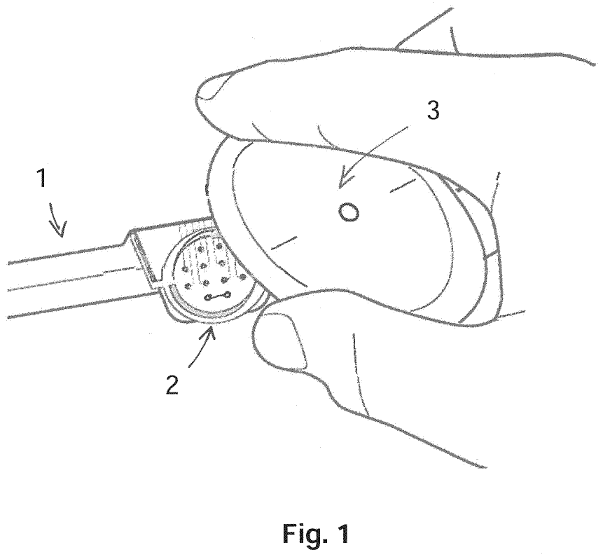

FIG. 1 an embodiment of a system in accordance with the invention comprising a patch having a connector and a sensor unit in a perspective representation in which the sensor unit has not yet been connected to the connector;

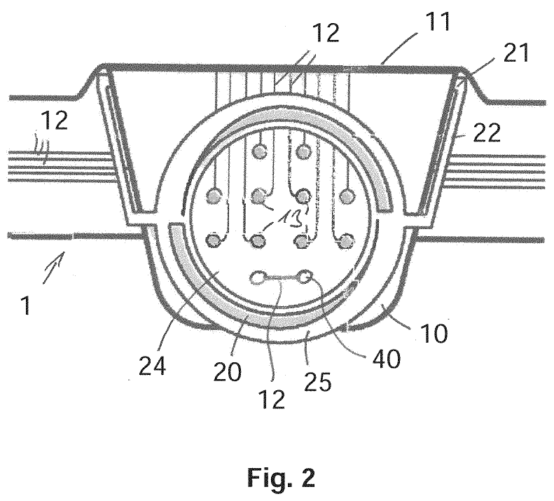

FIG. 2 a plan view of the second embodiment of a connector attached to a tab of the patch in the embodiment;

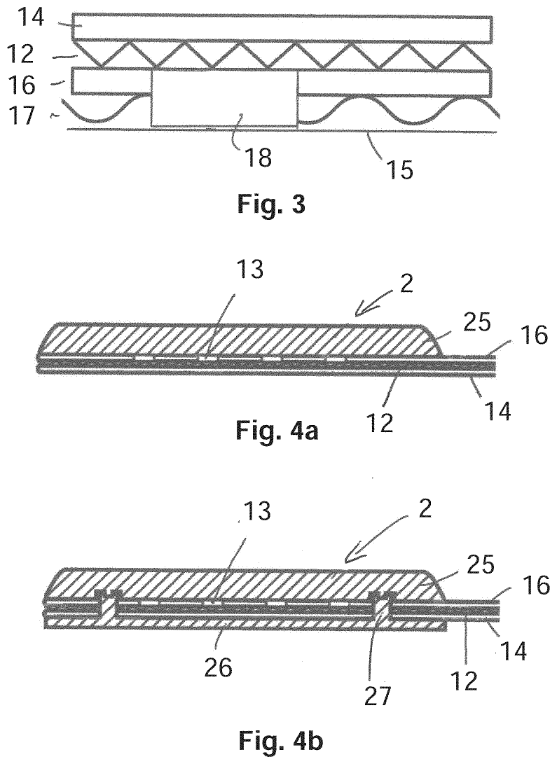

FIG. 3 a sectional view through an embodiment of the patch perpendicular to the plane of the patch in a region having an electrode;

FIG. 4a a sectional view through the embodiment of the patch perpendicular to the plane of the patch having a connector adhesively bonded to the patch;

FIG. 4b a sectional view through the embodiment of the patch perpendicular to the plane of the patch having a connector fastened to the patch via counter-element; and

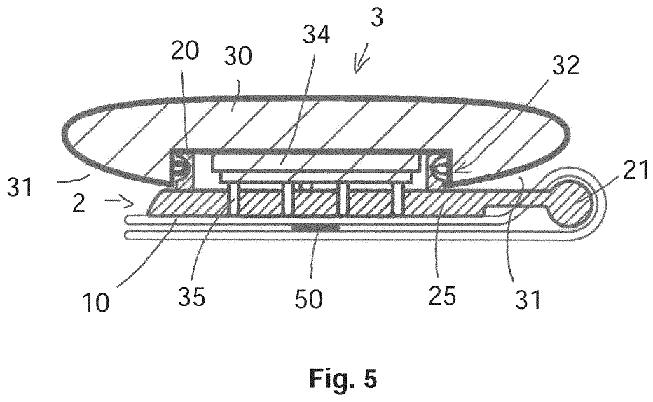

FIG. 5 a sectional view of the embodiment of a system in accordance with the invention comprising a patch having a connector and a sensor unit, with the sensor unit being connected to the connector, perpendicular to the plane of the patch.

The embodiment of the present invention described in FIGS. 1 to 5 is a portable system wearable on the body that serves the recording and preferably wireless transmission of biosignals. The embodiment implements all the independent aspects of the present invention in combination. The preferred embodiment of the independent aspects of the present invention explained in more detail in the following with reference to the embodiment is, however, also likewise part of the present invention respectively per se and without the other aspects.

The embodiment in accordance with the invention of the system in accordance with the invention shown in FIG. 1 comprises a measurement device in the form of a wireless sensor unit 3 and a patch 1 that is attached to the skin of the user. The patch 1 includes electrodes 18 for leading biosignals and electrical conductor tracks 12 that lead the biosignals from the electrodes 18 to the measurement device 3. The patch 1 is designed such that it is attached to the patient for application and is disposed of after use.

The sensor unit 3 is attached and held at the patch 1 via a connector 2. The connector 2 is designed such that a very simple attachment of the sensor unit 3 to the patch 1 is possible, preferably by one hand as a rotational movement. The connector 2 secures the mechanical long-term holding of the sensor unit 3 on the patch 1 during use, i.e. the sensor unit 3 is wearable on the body.

In addition, an electrical connection to transmit the biosignals from the patch 1 to the sensor unit 3 is also simultaneously established by the mechanical connection between the sensor unit 3 and the patch 1 established by the connector 2. The contacts can be implemented on sides of the sensor unit 3 e.g. in the form of spring-loaded contact pins 35 (so-called spring pins) that project from the sensor unit and preferably directly contact the leads 12 of the patch.

A primary application area of the system is the measurement, recording, and wireless transmission of medical biosignals and data in medical diagnosis, monitoring, and treatment. The data can be transmitted to portable end devices (smartphones, tablets, computers), to servers via radio nodes, or directly to databases and/or servers via a cellular radio and/or satellite network. The data can be evaluated directly on the patient by specialists or physicians in charge or in evaluation centers in real time or can be stored for later evaluation. Further applications for the system are in the fields of sports and wellness.

Preferred features of the embodiment that can be implement both per se and in combination will be initially briefly explained in the following with reference to the three components patch 1, connector 2, and sensor unit 3:

### Patch

The electrodes 18 and leads 12 are integrated into the patch 1. The adhesion of the patch thus intuitively always produces the correct positioning of the electrodes 18
The patch 1 has a folded-over tab 10 at which the connector 2 is arranged. This permits a very narrow design of the regions of the patch 1 adhering to the skin of the subject and/or an increased movability of the sensor unit 3 with respect to the patch 1
The one-time use of the patches 1 (disposable) provides a hygienic superiority of the system with respect to previous solutions since all the parts touching the patent are disposed of after use.

### Connector

The connector 2 enables a connection that can be established by one hand and/or is connected to the sensor unit via a rotational mechanism
On a correct attachment of the sensor unit 3 to the connector 2, a haptic feedback preferably takes place on the latching and/or on a successful connection “click”)
The connector 2 only establishes the mechanical connection and leaves contact regions 13 of the conductor tracks 12 accessible for the direct contact with the sensor unit 3, in particular by one or more cutouts 24
The connector is of simple construction and can be manufactured as an injection molded part. The costs for the disposables, that consists of the connector and the patch, are hereby reduced.

### Sensor Unit

A housing 30 of the sensor unit 3 is designed as wearer friendly, preferably with a rounded, convex shape of the housing surface.
The electrical contact takes place directly to the patch, in particular via spring-loaded contact pins 35 (so-called spring pins) projecting from the sensor unit.

The signals detectable by the embodiment of the system and application possibilities of the system will be described in the following in an overview manner.

### Signals

At least one, and preferably a plurality, of the following signals can be recorded directly as raw data by the embodiment of the system. Further parameters can be derived and calculated therefrom
ECG
EEG
EOG
EMG
Breathing frequency and activity, determination possible over a plurality of measurement methods or their combination:
via thoracic excursion (detection via motion sensor of the sensor unit)
via the impedance change of the thoracic ECG leads at the patient (by applying a voltage to two electrodes of the patch and measuring the resulting current by the sensor unit)
via periodic changes of the ECG vector (evaluation of the ECG by the sensor unit or a separate evaluation unit)
via strain gauges that are printed on the patch and that change their resistance on thoracic excursion and an associated sensor of the sensor unit. The strain gauges are conductively connected via conductor tracks to one or more contact regions of the patch and via them to contact elements of the sensor unit
breathing activity and breathing frequency
position data and/or rotation data and/or acceleration data (via the 9-axis motion sensor)

### Application Possibilities

The system preferably permits at least one and preferably a plurality of the following application possibilities:

a. 12 channel (or 16 channel) ECG, (e.g.) in a routine examination and/or on chest pain and/or unclear abdominal or thoracic complaints.
b. Exercise ECG (above all for use in a portable environment—e.g. when jogging, hiking, rowing, etc.).
c. Long-term ECG (e.g. with suspected cardiac arrythmias) or long-term EEG (with an application time of days or weeks, e.g. with suspected epilepsy).
d. As a telemetric solution and/or home care ECG usable by the patient himself.
e. Monitoring of ECGs and further vital parameters of patients in an ambulance and/or patient transport and/or in a clinic and/or in an ICU ward.
f. As an acute ECG (e.g. with unclear reduced vigilance, epilepsy diagnosis) or long-term EEG measurement (e.g. to measure sleep, epilepsy diagnosis)
g. Long-term home monitoring with life threatening arrythmias and/or for diagnosing cardiac arrythmias.

The sensor unit preferably has at least one interface for the wireless transmission of data, in particular a radio interface, in particular for near field communication such as Bluetooth (2.0, 4.0/smart, or 5.0), wireless LAN and/or NFC and/or a cellular radio data interface, for example via LTE, UMTS, and/or GSM. The sensor element can furthermore have a cabled interface for data transmission, for example via a USB interface.

In a first variant, the biosignals can be transmitted as raw data. In a second variant, the biosignals can be evaluated by the sensor unit and data can be transmitted on the basis of the evaluation.

One or more of the following solutions can be implemented for the transmission and/or processing of the biosignals.

a.) Transmission of biosignals from the sensor unit via a wireless interface (e.g. Bluetooth (2.0, 4.0/smart, or 5.0), wireless LAN, NFC, or cellular radio network) to a portable end device and/or computer (direct visualization) from there via a wireless interface (cellular radio network/internet) to a server and/or database and/or computer center (accessible from here on portable devices and/or via the internet).
b.) Transmission of biosignals from the sensor unit via a wireless interface (e.g. wireless LAN and/or cellular radio network and/or the internet and/or satellite) to a server and/or database and/or computer center (accessible from there on portable devices and/or via the internet)
c.) Pattern recognition and/or analysis of the signals running in the sensor unit. On recognition of a striking pattern (e.g. arrythmia and/or epilepsy) alarm and/or message to patient's cellular phone and/or to the physician and/or to the data center
d.) From the sensor unit via a wireless interface (e.g. Bluetooth (2.0 or 4.0/smart, 5.0), wireless LAN, NFC or cellular radio network) (optionally via a portable end device and/or computer) to an internal data management system at the clinic or practice
e.) Recording and storing the data in the sensor unit for later transmission to computers (by cable and/or wireless interface (e.g. cellular radio network and/or wireless LAN) and evaluation (e.g. as a long-term ECG)

The components and aspects of the present invention will be described again in the following in detail with reference to the embodiment:

### Sensor Unit

The sensor unit includes one, more, and preferably all of the following components
battery and/or rechargeable battery
charging circuit
signal processing module for the individual biosignal channels (e.g. filter, integrated AD converter front end, amplifier)
position sensor, rotation sensor, and/or acceleration sensor (gyroscope, magnetoscope, accelerometer)
memory (e.g. flash, RAM)
micro-USB port for electrical charging and/or for transfer of data
processor (e.g. ARM cortex)
wireless radio interface (e.g. BT 2.0 and/or 4.0 and/or 5.0 and/or wireless LAN GSM)
LED and/or a plurality of LEDs as status indicators
housing

Due to the relatively small connector and/or its arrangement at the tab, the patch only has small rigid regions or no rigid regions at all. The patch can hereby fold and/or bend with the body surface on a movement of the subject. The comfort in wear is hereby considerably improved.

In a possible embodiment, the housing of the sensor unit can be splash proof and/or waterproof. The housing can comprise at least two housing halves, with the connection region between the two housing halves having a seal.

### Patch

The design of the patch is shown in FIG. 3. The patch comprises the following components:
carrier substrate (e.g. TPU or PET film; thickness e.g. 50-100 micrometers)
adhesive 17 on the lower side of the patch by which the patch is fastened to the skin
electrical leads 12, preferably printed on the carrier substrate, e.g. from ink containing Ag and/or AgCl or carbon particles (carbon)
electrodes 18, integrated in the patch, e.g. comprising a layer of hydrogel that is applied over the conductor track and that includes e.g. ions (e.g. NaCl) that transmits the biosignals from the body to the leads.
a protective liner 15 that covers the electrode points and the adhesive up to use
packaging in which the patch can be stored in the long term (for example a packing unit of plastic film, or a metalized film)
   The further regions of the patch that do not include any electrical leads comprise in a layer-wise design the components carrier substrate 14, adhesive 17, and protective liner 15 and serve to reinforce the fastening of the patch to the patient.

### Manufacture of the Patch:

### Provision of Carrier Material and Conductor Tracks

The carrier material 14 is printed to provide the conductor tracks, e.g. as a sheet or in roll format by means of screen printing and/or flexographic processes
For this purpose, conductive ink (e.g. containing Ag or carbon) is first printed and sintered on one side of the carrier material 14 (by exposure and/or drying or similar)
The parts of the carrier material 14 that do not have any electrode points in skin contact and that are not used as contact regions 13 for electrical contact with the sensor unit are covered by a protective layer 16 (e.g. biocompatible polymer)
An adhesive material 17 that fastens the patch to the skin of the patient is applied to the protective layer 16. The adhesive material forms an adhesive layer
The protective layer 17 and the adhesive layer 17 can optionally also be provided by the same material.

### Manufacture of the Electrodes:

The conductive ink is possibly chlorinated at the points that are free of protective material 16 and adhesive 17 and that represent the skin electrodes 18 or a conductive ink that contains chloride and/or is chlorinated is printed over the first ink
A conductive hydrogel or a substance containing ions can be applied to the electrode points. This is done e.g. by manual placement of an already finished hydrogel (“slab gel”) or by machine deposition of a liquid hydrogel.

### Manufacture of the Contact Regions:

The conductive tracks are planar at the points that are intended to form the contacts regions 13 of the conductive tracks 12. No protective material 16 or adhesive 17 is furthermore located in the region of the contact regions 13. In this respect, a single cutout can be present in the protective material 16 and adhesive 17 for every contact region 13 or a common cutout for all the contact regions 13.
The contact regions 13 can reinforced mechanically and/or with respect to their conductivity in a variant. This can be done e.g. by applying an additional conductive layer, for example of a conductive plastic, and/or by using a plurality of layers of conductive ink, e.g. in that the contact regions are overprinted with one or more further layers of the ink.
In this respect, at least two of the contact regions (40) can be electrically conductively connected to one another. On a connection of these contact regions of the patch to contact elements, in particular contact pins of the sensor unit (35) on a coupling of the patch, a circuit between the contact elements can hereby be closed.

### Connector

The connection between the sensor and the patch is secured by the connector of which an embodiment is shown in FIG. 2.

The connector is a mechanical connection element, e.g. of a polymer, that is attached to the side of the patch actually at the patient side that is now, however, the side remote from the patient due the folding over of the tab. The connector, for example, measures approximately 2-6 cm in diameter and 3-5 mm in height.

The connector 2 is adhesively bonded to the patch, see FIG. 4a, and/or is held by a counter-element 26 on the patch side actually remote from the patient that is, however, only at the patient side due to the folding over, see FIG. 7b. Webs 27 are provided for the connection between the connector 2 and the counter-element 26 and pass thorough the patch or past the margin of the patch and, for example, latch at the other element and/or by a snap-in fastening. The connector and the counter-element could also be adhesively bonded to the patch instead of the webs 27.

The counter-element 26 can be a plate that e.g. has a thickness between 0.5 mm and 2 mm, in particular a thickness of 1 mm. The counter-element can be manufactured from a biocompatible plastic or a cardboard. In both cases, the lower side of the base plate 25 of the connector is disposed on the side of the patch actually remote from the patient that is, however, only at the patient side due to the folding over.

The connector 2 establishes the mechanical connection between the sensor unit 5 and the patch 1, i.e. it fastens the sensor unit 3 to the patch 1. The mechanical connection of the sensor unit 3 to the patch 1 by the connector 2 defines the position of the sensor unit on the patch and thus the position of the contacts, in particular of the contact pins 35 of the sensor unit 3, with respect to the contact regions 13 of the conductor tracks 12 on the folded-over tab 10. It is hereby ensured that the contact pins 35 of the sensor unit are correctly positioned and contacted.

As can in particular be seen from FIG. 5, the contact of the contact regions 13 of the leads 12 of the patch takes place directly by the contact pins 35 of the sensor unit. For this purpose, the connector has one or more cutouts 24 in the region of the contact regions 13, through which cutouts 24 the contact pins 35 pass and can contact the contact regions 13 on the patch.

In this respect, a separate cutout 24 can be provided in the connector for every contact region 13, as is shown in the first embodiment in FIG. 2. In a preferred embodiment, the connector, however, only has a common cutout 24 for all the contact region 13, for example in the form of an opening in a base plate 25 of the connector by which the connector lies on the patch. This design is shown in the second embodiment in FIG. 4. In this case, the connector preferably has the shape of a ring that surrounds the electrical contact regions 13 of the patch. The two embodiments in FIGS. 2 and 4 are otherwise identical.

To simplify the contacting of the contact regions 13 of the patch by the contact pins 35 of the sensor unit, the connector can have a counter-element 26, such as is shown in FIG. 4b. The patch can be supported on the counter-element at least in the region of the contact regions 13 of the patch so that the pressing force of the contact pins 35 on the patch is increased and a bulging of the patch in this region is avoided. In a first variant, the counter-element can be designed as of plate shape with a flat surface in the region of the contact regions 13 of the patch. In a second variant, the counter-element can have an elevated portion on its surface in the region of the contact regions 13 of the patch and the contact regions can be pressed through it into the cut-out 4 in the direction toward the sensor unit. The contact pins of the sensor unit hereby have to project less far out of the housing 30 of the sensor unit to contact the contact regions 13.

The connector 2 serves the simple and intuitive attachment of the sensor unit 3 to the patch 1 for the user. The use of one hand is sufficient to attach the sensor unit to the connector. The design of the connector makes possible the attachment via haptically intuitive elements so that the attachment can even take place without direct visual contact.

The connector is designed in the embodiment such that the attachment of the sensor unit 3 is executed by a rotational movement, e.g. clockwise. This rotational movement can comprise an angle of rotation of 10-180°. The sensor unit 3 is first positioned in a defined, preferably marked, first rotational position. The markings can be defined either optically, e.g. lines, or mechanically, e.g. by surface structures and/or surface arching, and make the clear positioning of the sensor unit in the first position on the connector possible for the user.

The design of the connector is shown in more detail in FIGS. 2 and 5. The connector has a base plate 25 by which it is arranged at the surface of the patch. A projecting annular region 20 is provided on the base plate and forms the mechanical connection region for connection to the sensor unit 3.

Arms 21 at which the web region 21 is arranged are provided at the base plate 25 in the embodiment. Alternatively, the function of the web region 21 could also be taken over by a marginal region of the counter-element 26 shown in FIG. 4b. This marginal region is preferably rounded for this purpose.

A guide in the form of a groove extending in the peripheral direction can be provided at the outer and/or inner periphery of the annular region 20. A cutout can be provided as a latching device at the end of the guide. Cutouts extending in the axial direction can furthermore be provided that lead to the start of the guide extending in the peripheral direction. Alternatively or additionally, an internal and/or external thread can be provided at the annular region 20.

In a possible embodiment, the connection between the sensor unit and the connector is splash proof and/or waterproof. This has the advantage that the patients/users can shower with it and perspiration would not cause any artifacts.

The housing can have a sealing element that cooperates with a sealing surface of the connector. The sealing element is here preferably pressed onto the sealing surface in the connected state. This can be achieved, for example, by an extent of the guide that has an axial offset over the extent in the peripheral direction so that the locking elements 33 exert a force in the axial direction onto the connector in the second rotational position. The base plate 25 can, for example, serve as a sealing surface and cooperates with a sealing element, for example, a sealing ring arranged on a housing edge.

The connector can be adhesively bonded to the surface of the patch, with the sealing surface of the connector completely surrounding the electrical connection region such that the electrical connection region is outwardly completely sealed between the sensor unit and the patch.

### Further Functions of the Sensor Unit

In the possible embodiment, two or more contact regions (40) of the patch are conductively connected. They are in particular connected to one another by a conductor track of the patch. Such a connection by a conductor track in the sense of the present invention is also present when the two contact regions are connected. A circuit can be closed by the two contact regions (40) of the patch that are conductively connected and 2 contact elements of the sensor unit (35) can, for example, be conductively connected to one another. This circuit can be used so that the sensor unit automatically switches on with a connection to the connector and the closing of the circuit resulting therefrom. A manual switching on by the user and the use of an On/Off button hereby becomes superfluous. A switching off of the sensor unit can equally take place by opening the circuit on the separation of the connection of the patch and the sensor unit. The recording of the biosignals preferably starts automatically on the switching on of the sensor unit.

Different further functions can be triggered in an automated manner by the use of patches having a different arrangement and/or connection of the conductive contact regions within the connector (24) and by the resulting different connections between the contact elements of the sensor unit. For example, different types of patches can be recognized and distinguished by the sensor unit by the closing of different circuits. The sensor unit can automatically execute different operating modes in dependence on the patch type. A sensor unit can, for example, distinguish between the operating mode recording of an EEG (higher sampling rate and resolution) and an ECG (lower sampling rate, lower resolution) by recognition of the patch type. In another example, the sensor unit can distinguish between the operating mode long-term ECG and 12 channel ECG.

In an embodiment, the sensor unit has a specific number of electrically conductive contact elements (35). If this number is higher than the number of the contact regions (13) present on the patch, input measurement channels of the sensor unit can remain “open” i.e. not connected to a closed circuit. The sensor unit only detects a noise signal on these channels that is recognized by analysis software of the incoming signals running in the sensor unit in that, for example, the amplitudes and frequency portions of the signal are analyzed. The sensor unit can thereby trigger an automatic switching off of the open, not connected measurement channels. A sensor unit with a total of 4 measurement channels can, for example, hereby be combined as desired with different patches that contain between 1-4 measurement channels in that it respectively automatically selects the number of measurement channels present. The sensor unit can in this manner also recognize a loss of the signal of an electrode during use, e.g. by release from the patient, and can likewise switch off this electrode from the measurement or can reduce the sampling frequency of the measurement at this electrode.

1. A system for detecting biosignals having a sensor unit and a patch that can be attached to a body and has electrodes and conductor tracks, wherein the sensor unit and the patch are connectable to one another via a connector arranged at the patch such that an electrical connection is established and the sensor unit is held at the body via the patch, wherein the sensor unit has a plurality of contact elements for an electrical contact of the connector and/or of the patch,
wherein
at least two contact elements of the sensor unit are conductively connected to one another via a conductor track of the patch by the connection of the patch to the sensor unit.

2. The system in accordance with claim 1, wherein the sensor unit recognizes that it has been connected to a patch and automatically switches itself on as a response.

3. The system in accordance with claim 2, wherein the patch and the sensor unit are set up such that a circuit of the sensor unit is closed by the connection of the patch to the sensor unit.

4. The system in accordance with claim 2, wherein a recording of signals starts automatically when the sensor unit recognizes that it has been connected to a patch; and/or wherein the recording of signals ends as soon as the sensor unit recognizes that it is no longer connected to a patch.

5. The system, for detecting biosignals having a sensor unit and a patch that can be attached to a body and has electrodes and conductor tracks, wherein the sensor unit and the patch are connectable to one another via a connector arranged at the patch such that an electrical connection is established and the sensor unit is held at the body via the patch, wherein the sensor unit has at least one first sensor that is connected to the electrodes of the patch by the connection of the patch to the sensor unit and detects biosignals by means of these electrodes,
wherein
the sensor unit furthermore has at least one second sensor for detecting position data and/or rotation data and/or acceleration data, with user inputs being recognized by evaluation of the data of the second sensor.

6. The system in accordance with claim 5, wherein the sensor unit has at least one operating mode in which the data of the first and second sensors are used together for monitoring a patient; and/or wherein the data of the second sensor are used to recognize artifacts in the data of the first sensor.

7. The system in accordance with claim 1, wherein the sensor unit has at least one first sensor that is connected to the electrodes of the patch by the connection of the patch to the sensor unit and detects biosignals by means of these electrodes, with the system having a function for detecting breathing activity.

8. The system in accordance with claim 7, wherein a detection of the breathing activity takes place by at least one of the following methods: detecting an impedance change of ECG leads by means of the patch; detecting periodic changes of an ECG vector by means of the patch; detecting the data of at least one strain gauge integrated in the patch; detecting a movement of a thorax by the second sensor arranged in the sensor unit to detect position data and/or rotation data and/or acceleration data.

9. The system in accordance with claim 1, wherein the sensor unit has at least two different operating modes that work with different patches connectable to the sensor unit.

10. The system in accordance with claim 9, wherein the sensor unit automatically recognizes the desired operating mode with reference to the patch connected to it, with the sensor unit having a plurality of contact elements for electrical contact with the patch, with the sensor unit recognizing the desired operating mode by evaluation of signals at the contact elements.

11. The system in accordance with claim 9, wherein the operating modes and/or patches differ at least with respect to a number and/or a geometrical arrangement of the electrodes; and/or wherein the operating modes differ by the type of detected biosignals;
and/or wherein the operating modes differ by a duration and/or frequency and/or accuracy of the detection of the biosignals and/or the type of the evaluation and/or storage and/or transmission of the detected data.

12. The system in accordance with claim 1, wherein the sensor unit can work with at least two different patch types and automatically recognizes the respectively connected patch type.

13. A sensor unit for a system in accordance with claim 1.

14. A patch for detecting biosignals, having electrodes and conductor tracks, wherein the patch has a connector via which it is connectable to a sensor unit such that an electrical connection is established and the sensor unit is held at a body via the patch, wherein the patch and/or the connector has/have a plurality of contact surfaces for the electrical contact with contact elements of the sensor unit, with at least two contact surfaces being conductively connected to one another.

15. A set of at least two different patches in accordance with claim 14 that are connectable to the same sensor unit for a system in accordance with claim 1.

16. The system in accordance with claim 10, wherein non-used contact elements are being recognized by the evaluation and/or the type of applied biosignals being recognized.

17. The system in accordance with claim 3, wherein the sensor unit recognizes that it has been connected to the patch by recognizing that the circuit of the sensor unit is closed by the connection of the patch to the sensor unit and automatically switches itself on.

18. The system in accordance with claim 3, wherein the sensor unit comprises a plurality of contact elements for the electrical contact of the connector and/or the patch, and the circuit is closed in that at least two contact elements of the sensor unit are conductively connected to one another by the conductive track of the patch.

19. The system in accordance with claim 1, further comprising an evaluation unit to which signals of the first and/or second sensors detected by the sensor unit and/or stored by the sensor unit are transmitted for evaluation.
